(19) United States
(12) Patent Application Publication
(10) Pub. No.: US 2008/0065216 A1
(43) Pub. Date: Mar. 13, 2008

(54) Artificial intervertebral disc

(75) Inventors: R. John Hurlbert, Calgary (CA); Stephan J. Duplessis, Calgary (CA); Lali Sekhon, Reno, NV (US)

Correspondence Address: Howard M. Ellis;SIMPSON & SIMPSON, PLLC, 5555 Main Street, Williamsville, NY, 14221, US

(73) Assignee: KINETIC SPINE TECHNOLOGIES, INC., Calgary (CA)

(21) Appl. No.: 11/978,872

(22) Filed: Oct. 30, 2007

Related U.S. Application Data
(63) Continuation of application No. PCT/CA2006/000677, filed on May 2, 2006.
(60) Provisional application No. 60/594,732, filed on May 2, 2005.

(51) Int. Cl. A61F 2/44

(52) U.S. Cl. 623/17.13; 623/17.15

(57) ABSTRACT

An artificial intervertebral disc for implantation between two adjacent vertebrae includes an outer casing, an inner casing contained within the outer casing, and a resilient nucleus contained within the inner casing. The inner and outer casings are formed, respectively, by two interlocking members. The resilient nucleus is biased against the two members forming the inner casing, thereby elastically separating same. The artificial disc includes various resistance means for restricting and limiting the range of rotational and translational motion between

### CROSS REFERENCE TO PRIOR APPLICATIONS

The present application is a Continuation of PCT application no. PCT/CA2006/000677, filed May 2, 2006, which claims priority from U.S. application No. 60/594,732, filed May 2, 2005. The entire disclosures of these applications are incorporated herein by reference.

### FIELD OF THE INVENTION

The present invention relates to the field of spinal implants and, more particularly, to implants comprising intervertebral disc replacements that provide dynamic spinal stabilisation.

### DESCRIPTION OF THE PRIOR ART

The spine is a complicated structure comprised of various anatomical components, which, while being extremely flexible, provides structure and stability for the body. The spine is made up of vertebrae, each having a ventral body of a generally cylindrical shape. Opposed surfaces of adjacent vertebral bodies are connected together and separated by intervertebral discs (or “discs”), comprised of a fibrocartilaginous material. The vertebral bodies are also connected to each other by a complex arrangement of ligaments acting together to limit excessive movement and to provide stability. A stable spine is important for preventing incapacitating pain, progressive deformity and neurological compromise.

The anatomy of the spine allows motion (translation and rotation in a positive and negative direction) to take place without much resistance but as the range of motion reaches the physiological limits, the resistance to motion gradually increases to bring the motion to a gradual and controlled stop.

Intervertebral discs are highly functional and complex structures. They contain a hydrophilic protein substance that is able to attract water thereby increasing its volume. The protein, also called the nucleus pulposis is surrounded and contained by a ligamentous structure called the annulus fibrosis. The main function of the discs is load bearing and motion. Through their weight bearing function, the discs transmit loads from one vertebral body to the next while providing a cushion between adjacent bodies. The discs allow movement to occur between adjacent vertebral bodies but within a limited range thereby giving the spine structure and stiffness.

Due to a number of factors such as age, injury, disease etc., it is often found that intervertebral discs lose their dimensional stability and collapse, shrink, become displaced, or otherwise damaged. It is common for diseased or damaged discs to be replaced with prostheses and various versions of such prostheses, or implants, as are known in the art. One of the known methods involves replacement of a damaged disc with a spacer into the space occupied by the disc. However, such spacers also fuse together the adjacent vertebrae thereby preventing any relational movement there-between.

More recently, disc replacement implants that allow movement between adjacent vertebrae have been proposed. Examples of some prior art implants are provided in the following U.S. patents: U.S. Pat. No. 5,562,738 (Boyd et al.); U.S. Pat. No. 6,179,874 (Cauthen); and U.S. Pat. No. 6,572,653 (Simonson).

Unfortunately, the disc replacement (i.e. implant) solutions taught in the prior art are generally deficient in that they do not take into consideration the unique and physiological function of the spine. For example, many of the known artificial disc implants are unconstrained with respect to the normal physiological range of motion of the spine in the majority of motion planes. Although some of the prior art devices provide a restricted range of motion, such restrictions are often outside of the normal physiological range of motion; thereby rendering such devices functionally unconstrained. Further, the known unconstrained implants rely on the normal, and in many cases diseased structures such as degenerated facets, to limit excessive motion. This often leads to early facet joint degeneration and other collateral damage to spinal components.

In addition, many of the artificial discs known in the art do not provide mechanisms for minimising stress upon adjacent structures caused by sudden motions.

Thus, there exists a need for an intervertebral disc implant that overcomes at least some of the deficiencies in the prior art solutions. More particularly, there exists a need for a spinal implant that allows for the reconstruction of spinal structures while preserving motion and protecting the facet joints of the affected segment of the spine from accelerated degeneration.

### SUMMARY OF THE INVENTION

In one aspect, the present invention provides an implant for replacing intervertebral discs.

In another aspect, the invention provides an artificial intervertebral disc that allows adjacent vertebrae a range of motion about various axes. Such motion is limited to a predetermined range within which movement of adjacent vertebrae does not lead to deterioration of neighbouring spinal structural components.

In another aspect, the above-mentioned motion about various axes can be coupled to more closely simulate natural movement.

Thus, in one aspect, the invention provides an artificial intervertebral disc for implantation between first and second adjacent vertebrae of a spine, the disc comprising:

an outer casing comprising cooperating first and second shells and defining a first compartment, the first and second shells being relatively moveable with respect to each other;

an inner casing comprising a cup and a cooperating lid, the cup and lid being relatively moveable with respect to each other, the inner casing defining a second compartment; and,

a resilient nucleus;

wherein,

a) the lid and cup of the inner casing are sized to have one of the cup or lid received within the other of the cup or lid;

b) the inner casing is substantially contained within the first compartment of the outer casing; and,

c) the resilient nucleus is substantially contained within the second compartment of the inner casing, the nucleus being biased against the cup and lid for elastically separating the cup and lid.

### BRIEF DESCRIPTION OF THE DRAWINGS

The features of the invention will become more apparent in the following detailed description in which reference is made to the appended drawings wherein:

FIG. 1 is a schematic illustration of the range of motion of a vertebra.

FIG. 2 is a side cross sectional view of an embodiment of invention taken along the line I-I of FIG. 4.

FIG. 3 is an end cross sectional view of the embodiment of FIG. 2 taken along the line II-II of FIG. 4.

FIG. 4 is a cross sectional top view taken along the line III-III of FIG. 2.

FIG. 5 is a radiograph of a spine illustrating a side cross sectional view of the disc of FIG. 2.

FIG. 6 is a side cross sectional view of another embodiment of invention taken along the line V-V of FIG. 8.

FIG. 7 is an end cross sectional view of the embodiment of FIG. 6 taken along the line VI-VI of FIG. 8.

FIG. 8 is a cross sectional top view taken along the line IV-IV of FIG. 6.

FIG. 9 is a side cross sectional view of another embodiment of the invention.

FIG. 10 is a radiograph of a spine illustrating a side cross sectional view of a disc according to another embodiment of the invention.

FIG. 11 is a radiograph showing the disc of FIG. 5 in top cross section.

FIG. 12 is a perspective view of the shells (or “endplates”) of the invention in accordance with another embodiment.

FIG. 13 is an end elevation of the shells of FIG. 12.

### DETAILED DESCRIPTION OF THE INVENTION

In the following description, the terms “superior”, “inferior”, “anterior”, “posterior” and “lateral” will be used. These terms are meant to describe the orientation of the implants of the invention when positioned in the spine. Thus, “superior” refers to a top portion and “posterior” refers to that portion of the implant (or other spinal components) facing the rear of the body when the spine is in the upright position. It will be appreciated that these positional terms are not intended to limit the invention to any particular orientation but are used to facilitate description of the implant.

The present invention provides artificial discs or implants for replacing intervertebral discs that are damaged or otherwise dysfunctional. The implants of the present invention are designed to allow motion between adjacent vertebral bodies but within normal limitations.

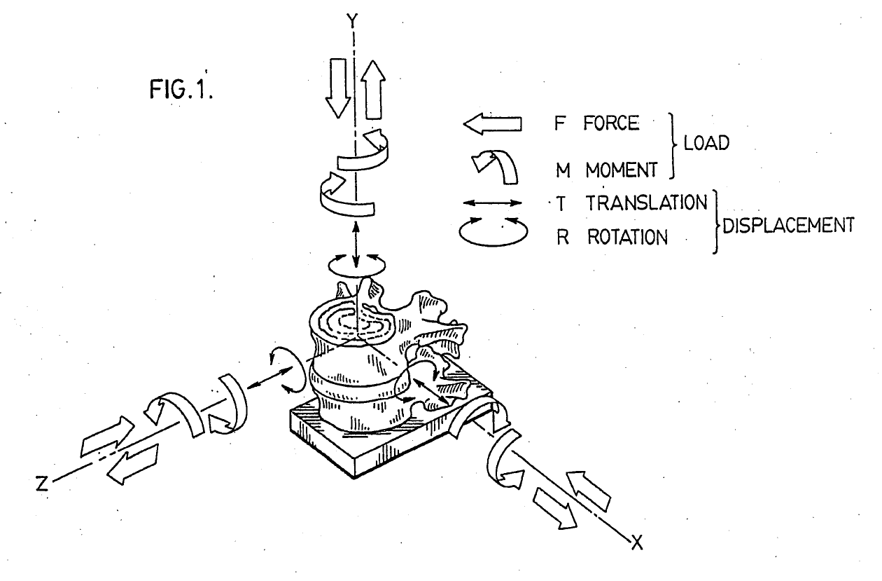

FIG. 1 illustrates the complexity of vertebral movement by indicating the various degrees of freedom associated therewith. In the normal range of physiological motion, vertebrae extend between a “neutral zone” and an “elastic zone”. The neutral zone is a zone within the total range of motion where the ligaments are relatively non-stressed; that is, the ligaments offer relatively little resistance to movement. The elastic zone is encountered when the movement occurs at or near the limit of the range of motion. At this zone, the visco-elastic nature of the ligaments starts providing resistance to the motion thereby limiting same. The majority of everyday motion occurs within the neutral zone and only occasionally continues into the elastic zone. Motion contained within the neutral zone does not stress soft tissue structures whereas motion into the elastic zone will cause various degrees of elastic responses. Therefore, in the field of spinal implants in particular, by restricting motion to within the neutral zone, stresses to adjacent osseous and soft tissue structures will be minimised. For example, such limitation of movement will reduce facet joint degeneration.

In general terms, the present invention provides a spinal implant for replacing intervertebral discs. The implant of the invention is generally comprised of various interlocking sections that are moveable relative to each other and which contain a resilient, force-absorbing nucleus. The relative movement between the components of the disc of the invention includes various degrees of freedom but is limited to a specified range. The present invention provides an artificial spinal disc for the replacement of intervertebral discs in the spinal column. The present invention, as will be described further below, allows for unconstrained and partially constrained vertebral motions at the site of spinal column insertion. In particular, the artificial disc of the invention provides for rotational, flexion, extension and lateral motions that are similar to normal movements in the neutral and elastic zones (i.e. the movements associated with a normal or intact disc). In addition, the device of the invention also allows various combinations of such motions, such as coupled motions. For example, the disc of the invention can be subjected to flexion and translation, or lateral flexion and lateral translation, or flexion and rotation. Various other motions will be apparent to persons skilled in the art given the present disclosure.

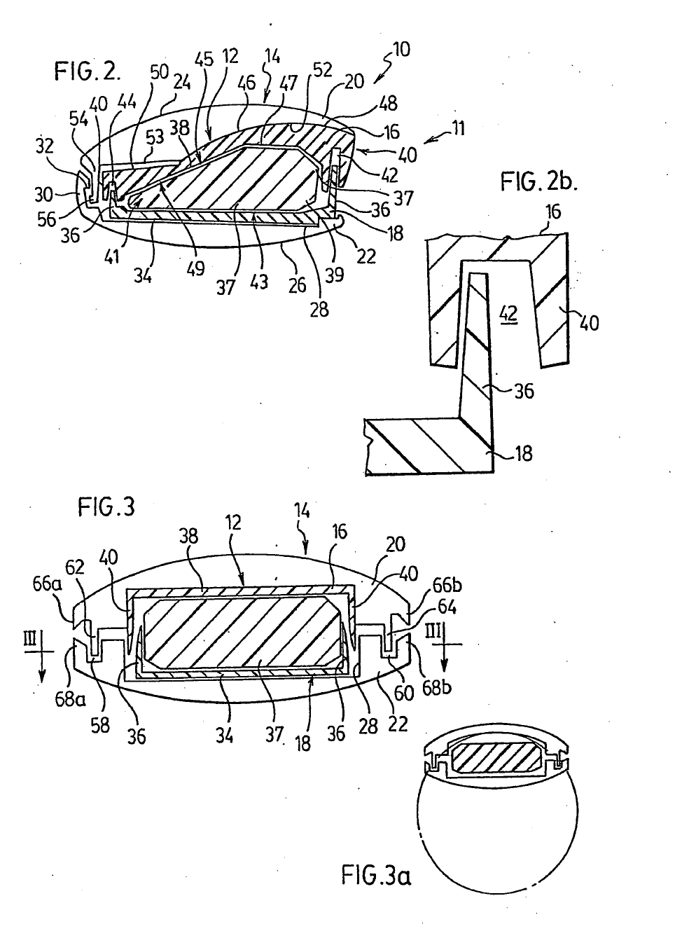

One embodiment of the spinal implant of the invention is illustrated in FIGS. 2 to 4. In the embodiment illustrated in these figures, the implant 10 is comprised of two overlapping casings, an internal casing 12 that is substantially enveloped by an external casing 14. As shown in the figures and as will be understood by persons skilled in the art, the term “substantially” as used in this context means that the inner casing 12 is for the most part surrounded by the outer casing 14 but that some portion of the inner casing may be exposed (i.e. not enveloped). Each of the casings 12 and 14 are comprised of two co-operating sections. The anterior and posterior ends of the implant are shown at 11 and 13, respectively. As shown, the inner casing 12 is comprised of a superior section or lid 16 (or “superior annulus”) and an inferior section or cup 18 (or “inferior annulus”). Similarly, the outer casing 14 is comprised of a superior section or superior shell 20 (or “superior endplate”) and an inferior section or inferior shell 22 (or “inferior endplate”). These sections are discussed further below. It is noted that although the term “annulus” is used throughout this description, it is done purely as a matter of convenience and this term is not meant to indicate that the lid 16 or cup 18 must have a fenestration, although this may be possibility in one embodiment of the invention.

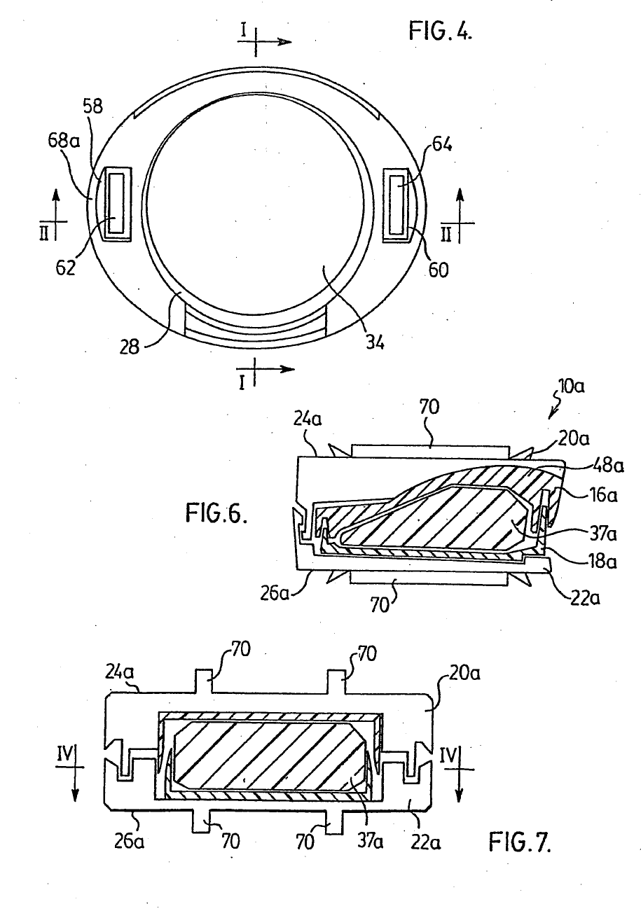

As illustrated in FIGS. 2 to 4, the outer casing 14 has a generally ovoid or ellipsoid shape when viewed in the superior aspect (i.e. FIG. 4) with a major axis extending laterally and a minor axis extending anteriorly-posteriorly. The superior and inferior shells, or endplates 20 and 22 of the outer casing have convexly curved external surfaces 24 and 26, respectively. In one embodiment, the external surfaces 24 and 26 are provided with a partial spherical curvature. Such curvature offers various advantages. For example, a curved configuration provides an increased surface area in contact with adjacent bone structures thereby promoting bony in-growth. In addition, the curved outer structure of the artificial disc of the invention maximises occupation of the intervertebral disc space once implanted.

In addition to the provision of increased surface area, the outer surfaces of the superior and inferior shells may be provided with one or more other promoters for bony in-growth. Such factors may be physical and/or chemical in nature. For example, the outer surface may be provided with a plurality of holes or pins and the like to which bone can attach. Alternatively or in combination, the outer surfaces of the shells may be provided with chemical bone growth components. These and other bone growth factors will be known to persons skilled in the art.

The inferior shell 22 (or inferior endplate) of the outer casing 14 includes a generally circular recess 28 (or “discoid recess”) for receiving the cup or inferior annulus 18. As illustrated in FIGS. 2 and 3, the recess 28 is dimensioned to be slightly larger than the cup 18. The posterior end of the inferior shell 22 includes a generally upwardly extending flange 30 including a hook 32, the purpose of which will be explained below. In addition, the anterior end of the shell 22 is provided with a ledge or bump to inhibit extrusion of components contained with the disc, as discussed further below.

The cup 18 (or inferior annulus) of the inner casing 12 includes a generally circular base 34 with a generally upwardly extending sidewall 36. As shown in FIG. 2, the sidewall 36 preferably has a greater height at the anterior end and a shorter height at the posterior end.

The base 34 of the cup 18 is adapted to receive and contain a resilient nucleus 37, which is discussed further below. The nucleus 37, according to the embodiment of the invention shown in FIGS. 2 and 3, generally assumes the shape of a wedge shaped disc when contained in the device, the nucleus having a thick anterior portion 39 and a thin posterior portion 41. The inferior surface, or base 43 of the nucleus is generally flat. In the result, the superior or top surface 45 of the posterior section 41 of the nucleus comprises a ramp 45. As will be understood from the following disclosure, the nucleus 37 may be manufactured with such a wedge shaped form or may simply comprise a general disc-shaped resilient body that, when contained within the disc cavity (as discussed further below), assumes the shape of the cavity.

The lid 16 (or superior annulus) of the inner casing 12 is adapted to fit over the cup 18. The lid 16 comprises a generally circular cover 38 having, as shown in FIG. 3, a radius slightly larger than that of the cup 18. The cover 38 is provided with a generally downwardly extending sidewall 40 that overlaps the sidewall 36 of the cup 18. As shown in FIG. 3, the recess 28 is sized to accommodate the diameter of the sidewall 40 of the cover 38. Moreover, it is noted that the recess 28 is also sized to be deep enough to allow the sidewall 40 to at least partially be accommodated when the lid 16 and cup 18 are moved towards each other. As will be understood, the movement of the lid 16 towards the cup 18 will be limited when the sidewall 40 contacts the base of the recess 28. The anterior and posterior ends of the sidewall 40 are provided with recesses or grooves 42 and 44, respectively, for receiving tongues projecting from the sidewall 36 of the cup 18. As shown in FIG. 2, the grooves 42 and 44 are adapted to allow reciprocating up and down movement of sidewall 36 there-within and, as will be discussed below, to allow the lid 16 and cup 18 to rotate with respect to each other about a vertical axis. As illustrated in FIG. 2, in one embodiment, at least the anterior groove 42 and the associated tongue extending from the inferior annulus 18 are preferably angled towards the posterior direction. In another embodiment, both of the grooves 42 and 44 are angled in such manner. In addition, in one preferred embodiment the grooves 42 and 44 and the associated tongues are wedge shaped wherein, the mouth of each groove is the widest portion thereof. This angled and wedge-shaped tongue and groove combination minimises the shear stresses across the nucleus and serves to assist the compression of the nucleus 37.

FIG. 2b illustrates a variation in the sizing of the groove 42. As shown, in one aspect, the groove 42 of the lid 16 may be wider (or oversized) than the sidewall 36 of the cup 18. As will be understood, such difference in size will enable a degree of relative translational movement between the cup and lid. Thus, in this aspect, the disc would limit flexion but would allow some degree of ventral or dorsal translation. It will be understood that the degree of translational movement allowed will depend on the amount of clearance provided to the sidewall 36 within the groove 42. It will also be understood that a similar oversizing may be provided in the opposite groove 44 of the lid 16.

The superior external surface 46 of the lid 16 is provided with a convexly curved anterior portion 48 and a generally flat posterior portion 50 or “turtledeck”. The inferior surface of the lid 16 includes an anterior section 47 that is adapted to contain the thicker anterior portion 39 of the nucleus. The inferior surface of the lid also includes a posterior section 49 that is angled so as to contain the ramped surface 45 of the nucleus. Thus, the combination of the superior surface of the cup 18 and the inferior surface of the lid 16 forms a nucleus cavity, for containing the nucleus 37 there-within. As will be understood, during the application of a compressive force on the implant, the volume of the nucleus cavity will be reduced. For this reason, as shown in FIGS. 2 and 3, one embodiment of the invention requires the volume of the nucleus cavity, in the rest position (i.e. when no compressive force is applied) to be larger than the volume of the nucleus 37. In this way, upon application of a compressive force on the disc 10, the resilient nucleus 37 is allowed to deform to occupy the reduced nucleus cavity volume.

It should be noted that the position and radius of curvature of the curved portion 48 may be changed in other embodiments. For example, depending upon the range of motion desired, the curved portion may be placed further posteriorly. In addition, it will be understood that the radius of curvature of the curved portion 48 will also affect the range of motion offered by the disc of the invention. This is discussed further herein.

The superior shell 20 (or superior endplate) includes an inner surface having an anterior end provided with a concave surface 52 that is adapted to slide over the convex surface 48 of the lid 16 (as discussed further below). Similarly, the posterior end of the superior shell 20 inner surface is includes a generally horizontal surface 53 that is adapted to slide over the turtledeck 50 of the lid 16. As shown in FIG. 2, in the neutral position, the flat surface 53 of the superior shell 20 is slightly separated from the flat surface 50 of the lid 16. This arrangement serves to allow a range of downward movement of the superior shell until the surfaces 50 and 53 come into contact, at which point a “hard stop” is reached for such motion. Motion of this sort will occur during extension of the spine, that is a front to back movement, generally in the sagittal plane. As will be understood, the range of extension motion offered by the implant can be predetermined by engineering a specified clearance between the surfaces 50 and 53.

The posterior end of the superior shell 20 includes a generally downwardly extending flange 54 terminating with a hook 56. As illustrated in FIG. 2, hook 56 co-operates with hook 32 of the inferior shell so as to prevent the superior and inferior shells from becoming separated. This is achieved with each hook being directed in opposite directions and being placed in a facing arrangement. In the embodiment shown in FIG. 2, hook 32 faces anteriorly while hook 56 faces posteriorly. However, other arrangements will be apparent to persons skilled in the art. As noted in the figures, a certain amount of clearance is provided so as to allow the flanges 30 and 54 to move with respect to each other before the hooks 32 and 56 engage one another. Such arrangement allows the superior shell 20 to rise up to a limit when the hooks engage. As will be understood the combination of the flanges 30 and 54 provide a hinge between the superior and inferior shells at the posterior ends thereof. This function is described further below.

As shown in FIG. 3, the inferior shell 22 is provided with two slots 58 and 60 positioned on the lateral edges thereof. The slots 58 and 60 are adapted to receive appropriately positioned tabs 62 and 64 provided on the superior shell 20. The tabs 62 and 64 act as “stabilisers” and serve to limit relative rotation between the superior and inferior sections of the implant. Specifically, as illustrated in FIGS. 3 and 4, the slots 58 and 60 are sized to be larger than the tabs 62 and 64 thereby allowing some freedom of movement of the latter within the former. As shown in FIG. 3, the tabs 62 and 64 are also shorter than the depth of the respective recesses 58 and 60, thereby allowing a range of lateral tilting of the superior shell 20 with respect to the inferior shell (22). In this way, the co-operating tabs and slots provide two benefits. Firstly, they act as a limiter or “hard stop” for relative axial rotation between the shells or endplates 20 and 22. Such axial rotation hard stop is reached when the tabs contact the sidewalls of the respective slots. As will be understood, the angular range of rotational motion can be predetermined by appropriate sizing of the tabs and slots. Further, the combination of the tabs and slots serve to limit relative translational movement between the shells 20 and 22 in the sagittal (anterior-posterior) plane or the coronal (lateral or side to side) plane.

As also illustrated in FIG. 3, the lateral edges of each of the superior and inferior shells, or endplates 20 and 22 may be provided, respectively, with ledges 66a, 66b and 68a, 68b extending from the edge of the shells. Ledges 66a and 68a extend from the respective shells 20 and 22 and are directed towards each other so as to narrow the gap there-between. Ledges 66b and 68b are similarly arranged on the opposite end of the shells. The arrangement of the ledges 66a,b and 68a,b result in a “cow catcher structure on both lateral sides of the implant. Such cow catcher arrangement serves to minimise the degree of scar tissue formation after implantation of the invention and, therefore, minimises any potential reduction in the disc's range of motion resulting. As will be understood by persons skilled in the art, the reduction in scar tissue formation is the result of the “pincer” like action between opposing ledges 66a/68a and 66b/68b. Specifically, with normal motion of the disc, the opposing ledges contact each other thereby shearing off any scar tissue and cutting off blood supply thereto.

The nucleus 37 of the invention, as indicated above, comprises a resilient material. In one embodiment, such material comprises a hydrogel, which is a material known in the art. However, alternative materials may also be used for the nucleus. For example, the nucleus may comprise a combination of mechanical springs, or an alternative compressible material as would be known to an individual of skill in the art. Generally, the nucleus is made from resiliently compressible materials. As can be seen in FIGS. 2 and 3, and as discussed above, the nucleus serves to bias the inferior and superior sections of the artificial disc against each other. It will be understood that the nucleus serves to absorb compressive forces that cause the inferior and superior sections of the implant towards each other. As will also be understood, due to the resilient nature of the nucleus, the superior section of the disc essentially “floats” above the inferior section. More specifically, by having the superior shell 20 attached generally loosely to the inferior shell 22, it can be seen that the superior shell 20 essentially “floats” on the annulus structure (formed by the superior and inferior annulus sections 16 and 18). Thus, the annulus (16 and 18) and the nucleus 37 combine to form a “rotating platform” which supports the shells 20 and 22. As will be appreciated and as mentioned above, the nucleus 37 may be provided in any shape and will assume the shape illustrated in the figures when contained within the nucleus cavity.

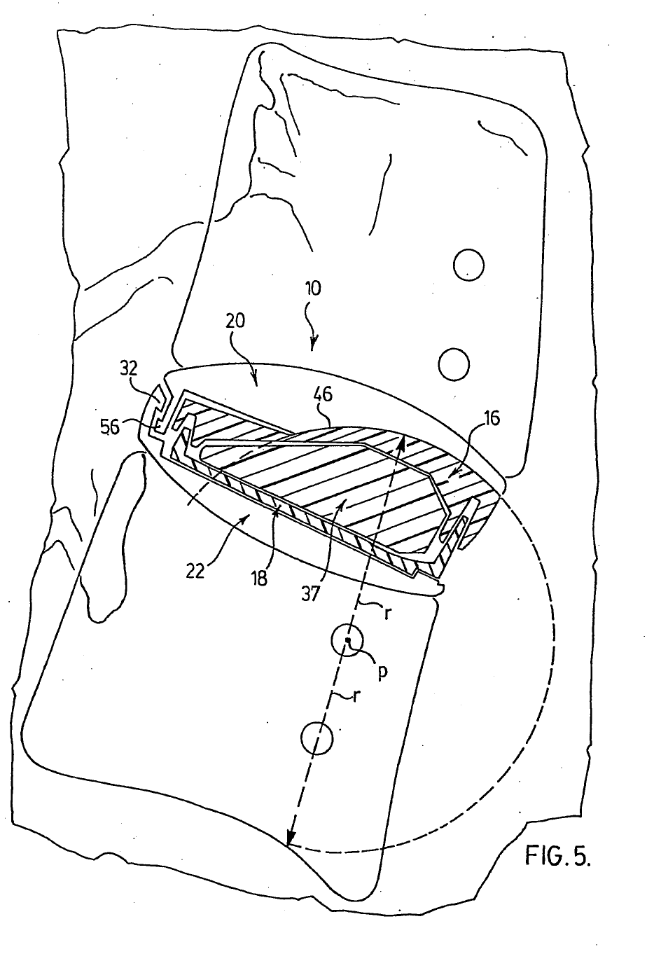

FIG. 5 illustrates the artificial disc 10 of the invention as it would appear when implanted within a spine. As will be understood, the disc 10 is implanted in the intervertebral space between adjacent vertebrae after excision of a damaged or diseased intervertebral disc. Also illustrated in FIG. 5 is the axis of rotation defining the curved portion 46 of the superior annulus 20. As can be seen, the curve provided on portion 46 is defined by an arc of a set radius “r” extending from an axis point “P”. As described herein, during flexion, the superior shell 20 first travels over the superior annulus 16, thereby traversing the arc of curved portion 46. Such movement occurs within the neutral zone of motion. As the movement continues into the elastic zone, the nucleus 37 begins to compress and offer some resistance to the motion, thereby forming a “soft stop” to the motion. Finally, as described above, the flexion movement is limited when the posteriorly located hooks (32 and 56) provided on the superior and inferior shells engage one another to form a “hard stop”. As indicated in FIG. 5, the axis of rotation P is located inferior to the artificial disk 10 and is located within the adjacent inferior vertebral body. Further axis point P is located near the anterior margin of such adjacent body, thereby providing an axis of rotation that is similar to the physiological axis of an intact, normal disk. As explained further below and as will be understood by persons skilled in the art, the location of axis point “P” and the length of radius “r” may be varied to provide the disc with different functional characteristics. For example, the axis point “P” may be positioned further posteriorly in the subjacent vertebral body.

FIGS. 6 to 8 illustrate another embodiment of the invention wherein elements common with the embodiment described above are identified with common reference numerals but with the letter “a” added for clarity. As with the embodiment described above, the artificial disc 10a illustrated in FIG. 6 is also comprised of a resilient nucleus 37a surrounded by superior and inferior annulus components, 16a and 18a, as well as superior and inferior shells 20a and 22a. The superior annulus 18a includes an anterior curved portion 48a over which is slidably provided the superior shell 20a.

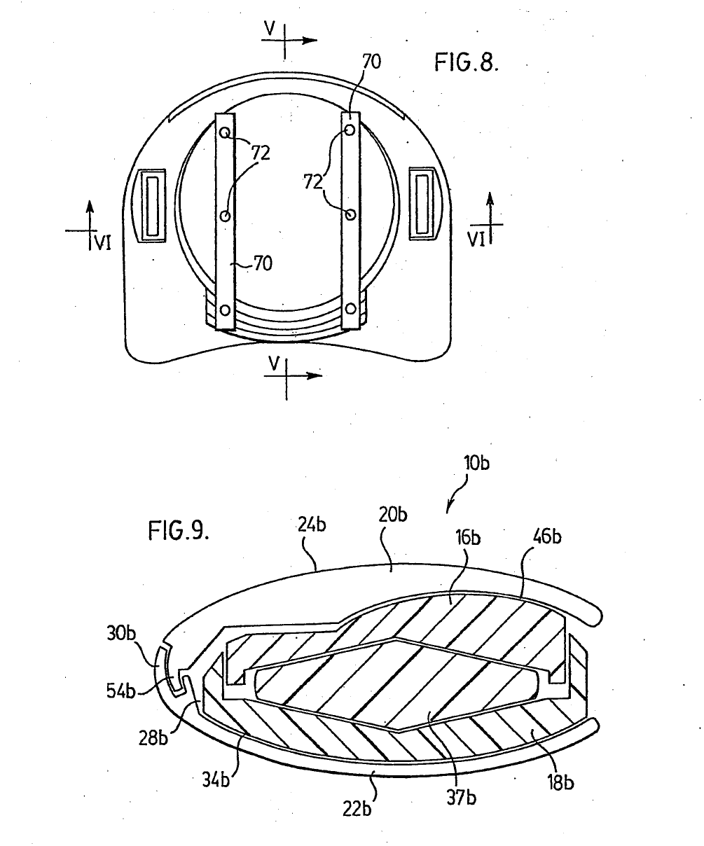

As can be seen, in the embodiment illustrated in FIG. 6 to 8, the disc 10a has a generally rectangular cross section with flat external surfaces 24a and 26a provided on shells 20a and 22a, respectively. Such a configuration may be preferred in situations where the discectomy site (where the implant is to be inserted) has a rectangular outline as opposed to a bi-concave site, wherein the previous embodiment would be preferred. In order to enhance implantation of the disc 10a, the external surfaces 24a and 26a are each provided with a pair of elongate ribs or “stabilising keels” 70. As shown in FIG. 8, the keels 70 are also preferably provided with a number of holes 72 through which (bone) anchoring screws can be inserted. It will also be understood that such screws may be used to anchor the artificial disc of the invention to other adjacent structures such as, for example, artificial vertebrae etc.

A further embodiment of the invention is illustrated in FIG. 9 invention wherein elements common with the embodiments described above are identified with common reference numerals but with the letter “b” added for clarity. In this embodiment, the inferior shell 22b includes a recess 28b that includes a concave curvature for receiving the inferior annulus 18b, the latter having a similarly curved inferior surface 34b. This arrangement allows the inferior annulus to slide over the inferior shell, particularly during bending in the coronal plane (i.e. laterally) or during axial rotation. The superior annulus 16b is provided with a curved anterior portion 46b as described previously, which facilitates flexion and extension movements (i.e. movements in the sagittal plane). As also illustrated, the nucleus of this embodiment includes a lateral cross section (taken generally along the sagittal plane) that is essentially “diamond” shaped wherein the thickest portion is provided in the centre of the nucleus and the anterior and posterior ends are thinner. Such a nucleus construction is particularly suited for axial loading.

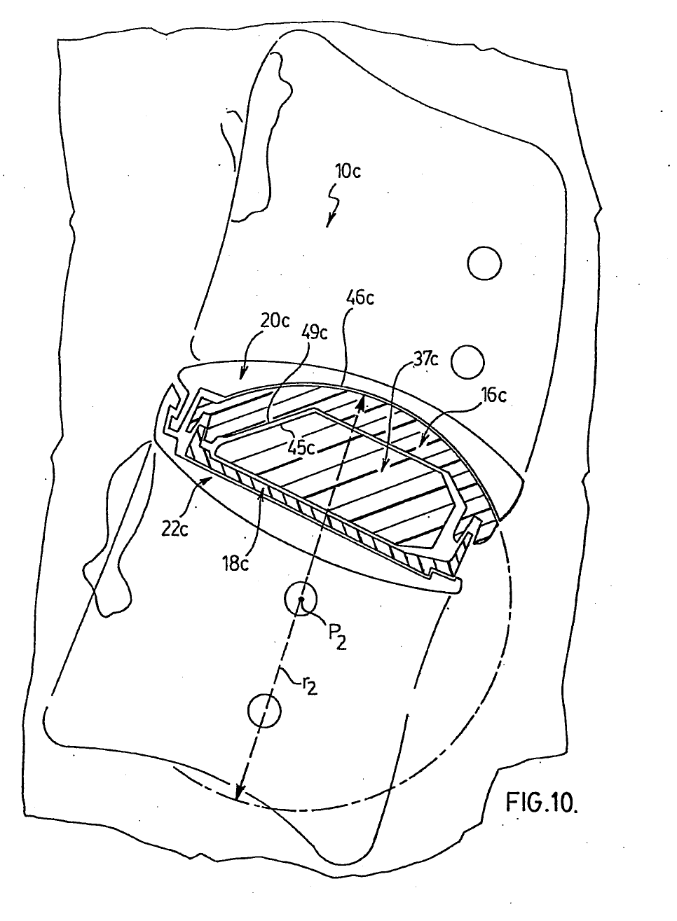

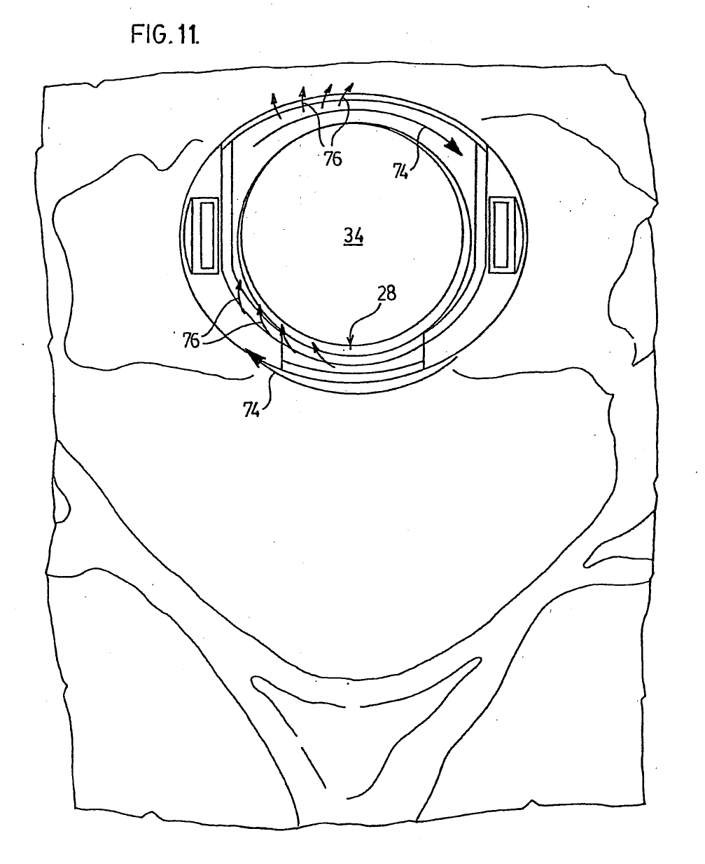

A further embodiment of the invention is illustrated in FIG. 10 wherein elements common with the embodiments described above are identified with common reference numerals but with the letter “c” added for clarity. In this embodiment, the curved portion 46c extends over essentially the entirety of the upper surface of superior annulus 16c. The curved portion 46c follows the arc of a curve having an axis of rotation P₂ and a radius of curvature r₂. As compared to FIG. 5, it is noted that the axis of rotation P₂ is still located within the inferior adjacent vertebral body but is further posterior. It will be understood, that the embodiments illustrated in FIGS. 5 and 10 will have use in different locations of the spine or in the same locations but for different purposes such as restoration of lordosis in a kyphotic segment. As noted in FIG. 10, the shape of the nucleus cavity differs with respect to the previously discussed embodiments to accommodate a nucleus 37c having a shorter and steeper ramp portion 45c.

FIG. 11 illustrates the artificial disc as shown in FIG. 5 but in a superior view. As seen, the base 34 of the inferior annulus is smaller than the discoid recess 28 provided in the inferior shell. As discussed above, and as further elaborated below, this difference in size permits translational movement of the inferior annulus with respect to the inferior shell. Such translational movement can occur in the sagittal and/or lateral (coronal) directions. Such translational movement is illustrated in FIG. 11. As shown, a rotational movement of the superior shell in the direction shown by the arrow 74 results in rotation of the inferior annulus as well. However, in addition, a translational movement of the inferior annulus also results, such translational movement being shown by the arrows 76.

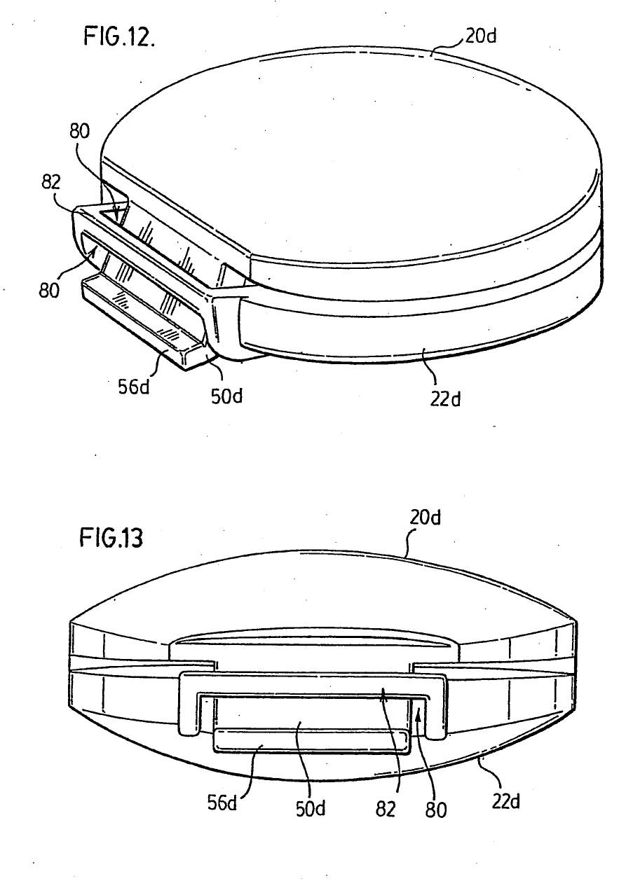

A further embodiment of the invention is illustrated in FIGS. 12 and 13 wherein elements common with the embodiments described above are identified with common reference numerals but with the letter “d” added for clarity. In this embodiment, a variation in the locking mechanism between the superior shell 20d and inferior shell 22d is shown. For the purpose of clarity, FIGS. 12 and 13 do not show other elements of the disc. As described above, one embodiment for the interaction mechanism between the shells 20 and 22 was the provision of flanges 30 and 54 each having interacting hooks to form a hinge between the shells. In the embodiment shown in FIGS. 12 and 13, such flanges and hook are replaced with a variant of the hinge mechanism comprised of a hook and slot assembly as shown. More specifically, in one aspect, the superior shell 20d may be provided, at their posterior ends, with a flange 50d which terminates in a hook 56d. However, the inferior shell 22d is provided with a slot 80 into which the hook 56d and flange 50d of superior shell 20d extend. As can be seen, the hook and slot assembly of FIGS. 12 and 13 function in essentially the same manner as the hook assembly of the previous figures. The slot 80 is preferably wider than the flange 50d so as to allow a degree of relative rotation between the superior and inferior shells about the hinge. In addition, as shown, the flange 50d is elongated so as to extend through the slot 80. In this manner, the flange 50d will remain within slot 80 while allowing for the shells 20d and 22d to be slightly separated. Such an arrangement would allow for the expansion of the inner core (i.e. the lid, cup and nucleus) but would still prevent overexpansion. Further, the flange 50d extends obliquely from the superior shell 20d thus allowing the superior shell to undergo flexion but with resistance as a result of such oblique configuration. Flexion will continue until the hook 56d contacts the upper bar 82 of the slot 80.

Summary of Features of the Invention

As discussed above, the artificial disc of the present invention includes various features, which will now be summarised. Firstly, in one aspect, the disc isolates axial rotation, lateral bending, and flexion/extension into component vectors and includes various structural components to accommodate such movements. In the result, the disc generally reproduces neutral zone and elastic zone movements associated with an intact disc along such individual component vectors. Further, the invention allows for unconstrained and partially constrained coupled movements making use of engineered end-points that prevent excessive or non-physiological movement. The fully constrained stop mechanisms (i.e. the “hard stops”) ensure that movement is not extended past the elastic zone.

In another embodiment, the disc of the invention may be generally wedge shaped in the sagittal plane so as to integrate with and promote a lordotic spine configuration. Such an implant may be used in cases where spinal re-alignment is sought. For example, the disc may have a larger height at the anterior end as compared to the height of the posterior end to provide the aforementioned wedge shape. Similarly, such a difference in height may also be provided between the lateral sides of the disc, that is in the coronal plane. This type of configuration may be used, for example, to correct a malalignment such as scoliosis.

The generally spherically curved external surfaces of the shells provide the disc of the invention with an ovoid curvature in the coronal plane. This structure maximises disc to bone surface area and thereby promotes bony ingrowth. Such structure also maximises prosthetic occupation of the disc space while stabilizing disc against bone after implantation. It will also be understood that the ovoid lateral curvature also maximises stability on floating annulus/nucleus complex in the coronal plane.

As will be appreciated by persons skilled in the art, the wedge shaped configuration of the nucleus and of the inner casing 12, in combination with the generally loose integration of the superior and inferior shells (20 and 22), facilitates the removal and replacement of the annulus/nucleus complex from an anterior approach. This is an important feature when the nucleus and/or annulus needs to be extracted after implantation for later revision or replacement.

As discussed above, the superior shell 20 associates loosely with the inferior shell 22 thereby allowing the superior shell 20 to float on the annulus and nucleus structures. Therefore, the annulus and nucleus serve to provide a “rotating platform” which supports the shells 20 and 22.

Vertically extending shell, or endplate stabilisers serve to provide a “hard stop” against excessive coronal or sagittal translation of the shells, while allowing moderate degrees of translation in these same planes between the endplate, annulus, and nucleus components. The shell stabilisers also provide a hard stop for axial rotation after a predefined amount of angular motion is achieved.

A “tongue and groove” relationship is provided between the superior and inferior annulus, which is achieved by the tongues extending from the inferior annulus being received within the anterior and posterior grooves 42 and 44, respectively, provided on the superior annulus. The preferred slanted or angled nature of the tongue and groove arrangement, in combination with the preferred wedge shaped design (as described above), serves to minimise translation (shear) across the nucleus while facilitating compression of the nucleus in flexion, neutral, or extension attitudes.

As illustrated in the figures, the surface areas of the base 43 of inferior annulus (or cup) 18 and the recess 28 of the inferior shell are relatively large in comparison with artificial disc. By maximising the surface areas of these components, it will be appreciated that any axial load on the disc is distributed over a greater area during all movements. In particular, such load distribution is achieved during flexion and extension movements, thereby minimising wear between the inferior annulus 18 and the inferior shell 22.

The posterior shell catch mechanism allows for angulation of its latch components to maintain locking competence during flexion and translation of the superior shell on the annulus/nucleus/inferior shell.

The floating configuration of the annulus/nucleus and superior/inferior shells transmits unconstrained axial loads through the nucleus even when the spine is not in a neutral position.

In other embodiments, the curved portion 48 of the superior annulus 16 may be moved more anterior or posterior to create different flexion/extension axes of rotation for different areas of the spine. In other embodiments, the radius of curvature of the curved portion 48 may be increased or decreased to create different flexion/extension axes of rotation.

The external surfaces of the superior and inferior shells may be curved or spherical (i.e. ovoid, elliptical) or straight (i.e. squared) for insertion into bi-concave or rectangular discectomy site at any area of the spine. The external surfaces may optionally be provided with anchoring ribs or keels for securing the disc to adjacent bone structures. The keels are preferably provided on the outer surfaces of the “squared” shells for increasing the stability of the prosthesis upon implantation. The keels, preferably provided in pairs on each surface, also serve to provide screw conduits for attachment of the artificial disc shells to an artificial vertebral body. Keels can also be used to run side to side (i.e. parallel to the coronal plane) particularly for implantation of discs from a lateral exposure.

In one embodiment, the superior shell may be larger in diameter, as taken in the sagittal plane (i.e. the anterior-posterior direction), than inferior shell so as to better approximate the “normal” condition.

In one embodiment, dimples or bumps may be provided on the anterior and posterior midline of the shell outer surfaces so that their alignment and positioning can be verified on x-rays.

In another embodiment, wedge shaped lordotic shells can be substituted for curved or squared shells to help realign the spine

The footprint of the disc is preferably maximised in both the coronal and sagittal planes to help eliminate subsidence. As will be understood, the size of the discs of the invention will vary to accommodate various sizes of discs in the normal spine.

The anterior end of the inferior shell and the discoid recess are provided with ledges to prevent anterior extrusion of the nucleus and/or the annulus once the prosthesis is implanted.

Other features of the invention will now be discussed with specific reference to directional movements.

1) Flexion and Extension

As indicated above, and as illustrated in FIG. 5, the axis of rotation of the curved outer surface of the superior annulus is preferably located in the adjacent vertebral body located below the artificial disc, and, more preferably, near the anterior margin of the vertebral body. Such an orientation mimics the physiological axis in an intact normal disc. As a result, for example, the tendency of the spine to fall forward into kyphosis is prevented and the spine instead maintains a lordotic orientation.

In other embodiments of the invention, the axis of rotation of the curve of the superior annulus may be positioned in other locations and/or the radius of curvature may be varied. For example in an alternate embodiment, the curved region of the superior annulus may be located further toward the posterior portion of the disc body in order to change the characteristics of spinal flexion and axial loading on the artificial disc. This can be seen in comparing FIGS. 5 and 10.

As will be apparent from the above discussion, neutral zone movement of the artificial disc of the invention, when in initial flexion, is provided by rotation of the superior shell over the superior annulus convex region. Elastic zone movement occurs with compression of the nucleus, which is necessitated by progressive distraction of posterior spinal elements during advanced flexion. Elastic zone movement in flexion unloads facets by rotating a superior facet away from an inferior facet of an adjacent vertebra without causing impaction of the facets or shear through them.

A “hard stop” in a flexion motion is provided by the posterior shell catch mechanism, or hooks after a predetermined amount of movement. Such a hard stop prevents excess motion beyond the elastic zone. The locking components of the posterior shell catch mechanism are preferably angled to maintain locking competence during flexion and translation. As will be appreciated, the compression of the nucleus prior to engagement of the hard stop (wherein the nucleus compression serves as a soft stop) serves to reduce the amount of wear on the catch mechanism.

The floating configuration of the superior shell on the superior annulus also allows for neutral zone movement during extension. Such movement narrows the gap or space above the turtledeck of the superior annulus. Compression of the superior shell horizontal region on the turtledeck during extension is transmitted to the nucleus providing elastic zone movement. The turtledeck also provides a hard stop after a predetermined amount of motion to prevent excessive movement.

The tongue and groove relationship of the superior annulus and the inferior annulus, in addition to the posterior shell catch mechanism, provide a hard stop in extension preventing excessive movement through the prosthesis after a predetermined amount of motion.

2) Rotation

The sidewalls of the superior shell constrain the superior annulus therewithin. Further, due to the fact that the inferior annulus is constrained by the superior annulus, it will be understood that any rotational movement of the superior shell forces the entire annulus (i.e. superior and inferior parts), and the resilient nucleus contained therein, to rotate with the superior shell against the inferior shell.

In a preferred embodiment, the discoid shape of the bottom of the inferior annulus integrates with the larger discoid recess in the inferior shell. This allows sagittal and lateral translation during twisting movements and, thereby eccentric rotation. As will be understood by persons skilled in the art, such movements serve to effectively mimic the physiological axis of rotation of normal discs. Further, the walls of the recess provided in the inferior shell also serve as hard stops for any excessive sagittal or lateral translation movement of the inferior annulus.

As explained above, the lateral shell stabilisers provide a hard stop for relative rotation of the superior shell and the inferior shell.

3) Lateral Bending

As indicated above, the superior annulus is adapted to fit over the inferior annulus whereby the inferior annulus is able to telescope inside the superior annulus. The space between the lateral walls of the superior annulus and the inferior annulus allows for lateral bending (bending in the coronal plane) with the eccentric compression of the nucleus. The neutral zone remains confined to neutral spinal alignment encouraging the spine to remain straight, avoiding a scoliotic attitude. The stabilisers provided laterally on the shells serve as a hard stop after a predetermined amount of lateral bending. It will be appreciated that such limitation prevents excessive lateral motion.

4) Coupling Motions

As described above, during flexion, the nucleus of the artificial disc compresses as the superior shell slides over the curvature of the superior annulus. Such movement causes the axis of rotation to descend with respect to the adjacent inferior vertebral body thereby mimicking the physiological relationships in the intact normal disc. Thus, the coupling of the movement of the superior shell over the curvature of the superior annulus and the flexion induced compression of the nucleus results in a gradual loading of the normal posterior elements in flexion until the hard stop is reached. As mentioned above, this combined movement reduces the wear on the posterior shell elements providing the hard stop.

The floating annulus/nucleus complex of the present invention, as described above, allows the aforementioned flexion/extension and axial rotation motions to be coupled with lateral bending thereby mimicking normal physiological movement.

Coupling of lateral angulation and lateral (coronal) translation with lateral bending occurs until the hard stop of the lateral shell stabilisers is encountered.

5) Compression

The tongue and groove arrangement provided on the anterior and posterior ends of the superior and inferior annuli provides stability and protects the resilient nucleus against translational shear. Further, such arrangement provides a defined limitation (i.e. a hard stop) on the degree of nucleus compression.

The generally trapezoidal shape of the resilient nucleus (when taken in sagittal cross section) allows maximum durability under loads of eccentric compression from directions other than true axial loading. As mentioned above, the nucleus cavity is designed to be larger than the nucleus itself. It will be understood that such extra space between the sides of the resilient nucleus and the superior annulus and the inferior annulus allows for lateral expansion during compression of the nucleus such as during axial loading of the disc.

The disc of the invention can be made with a variety of materials as will be known to persons skilled in the art. For example, the shells and annulus sections may be manufactured from steel, stainless steel, titanium, titanium alloy, porcelain, and plastic polymers. The nucleus may comprise mechanical springs (for example made of metal), hydraulic pistons, a hydrogel or silicone sac, rubber, or a polymer or elastomer material.

Although the invention has been described with reference to certain specific embodiments, various modifications thereof will be apparent to those skilled in the art without departing from the purpose and scope of the invention as outlined herein. The entire disclosures of all references recited above are incorporated herein by reference.

We claim:

1. An artificial intervertebral disc for implantation between first and second adjacent vertebrae of a spine, the disc comprising:
an outer casing comprising cooperating first and second shells and defining a first compartment, the first and second shells being relatively moveable with respect to each other;
an inner casing comprising a cup and a cooperating lid, the cup and lid being relatively moveable with respect to each other, said inner casing defining a second compartment; and,
a resilient nucleus;
wherein,
a) the lid and cup of said inner casing are sized to have one of said cup or lid received within the other of said cup or lid;
b) the inner casing is substantially contained within the first compartment of the outer casing; and,
c) the resilient nucleus is substantially contained within the second compartment of the inner casing, the nucleus being biased against the cup and lid for elastically separating said cup and lid.

2. The artificial disc of claim 1 wherein said outer casing is provided with one or more first restraining means to limit relative movement between said first and second shells.

3. The artificial disc of claim 2 wherein said inner casing is provided with one or more second restraining means to limit relative movement between said cup and lid.

4. The artificial disc of claim 3 wherein the first and second restraining means include stops to prevent said relative movement beyond a set point.

5. The artificial disc of claim 4 wherein the first shell of the outer casing is provided over the lid of the inner casing and wherein the first shell is slidable over the outer surface of the lid.

6. The artificial disc of claim 5 wherein the outer surface of said lid includes a convex portion and wherein said first shell includes an inner surface with a cooperative concave portion for receiving the lid convex portion.

7. The artificial disc of claim 6 wherein the convex portion of the lid is curved about the sagittal plane.

8. The artificial disc of claim 7 wherein the lid and cup of the inner casing are generally circularly shaped.

9. The artificial disc of claim 8 wherein the first and second shells of the outer casing include convex outer surfaces.

10. The artificial disc of claim 9 wherein the first and second shells are convex over both the coronal and sagittal planes.

11. The artificial disc of claim 10 wherein the outer surfaces of the first and second shells are generally spherical.

12. The artificial disc of claim 12 wherein at least a portion of the outer surfaces of the first and second shells are provided with physical or chemical bone growth promoters.

13. The artificial disc of claim 8 wherein the outer surfaces of the first and second shells include stabilizing members for anchoring the shells to adjacent vertebral bone structures when implanted.

14. The artificial disc of claim 13 wherein said stabilizing members include apertures for receiving bone anchoring screws.

15. The artificial disc according to claim 1 wherein said disc includes anterior, posterior and lateral ends and wherein the first and second shells of the outer casing are hingedly engaged at said posterior end.

16. The artificial disc of claim 15 wherein one of the first or second shells of the outer casing includes a first tongue at each lateral end and wherein the other of the first or second shells includes a complementary first groove at each lateral end, said first grooves being adapted to receive said first tongues.

17. The artificial disc of claim 16 wherein one of the lid or cup of the inner casing includes a second tongue at each of the anterior and posterior ends and wherein the other of lid or cup includes a complementary second groove at of the anterior and posterior ends, said second grooves being adapted to engage said second tongues.

18. The artificial disc of claim 17 wherein the grooves are wider than the tongues whereby the lid and cup are translationally moveable with respect to each other.
